(19) United States
(12) Patent Application Publication
(10) Pub. No.: US 2024/0358879 A1
(43) Pub. Date: Oct. 31, 2024

(54) DISPENSING SYSTEM FOR DISPENSING A FLUID INTO THE ATMOSPHERE

(71) Applicant: KSP HOLDING B.V., Lieshout (NL)

(72) Inventors: Bram VAN DE MOLENGRAAF, Lieshout (NL); Dennis Alexander Stefan VAN DUIJNHOVEN, Lieshout (NL)

(21) Appl. No.: 18/692,080

(22) PCT Filed: Sep. 19, 2022
(86) PCT No.: PCT/NL2022/050524

§ 371 Date: Mar. 14, 2024

(30) Foreign Application Priority Data
Sep. 23, 2021 (NL) 2029229

(51) Int. Cl. A61L 9/12; A61L 2/20; A61L 2/26

(52) U.S. Cl. CPC A61L 9/127; A61L 2/20; A61L 2/26; A61L 2202/25

(57) ABSTRACT

A dispensing system for dispensing a fluid into the atmosphere which includes a container for containing a liquid solution, a rigid support and a capillary fabric, wherein a first section of the support is positioned inside the container through an opening of the container and a second section of the support is positioned outside the opening of the of container, wherein the support is at least partially U-shaped or L-shaped by means of the second section of the support. The capillary fabric is attached around the first section and second section of the support, such that the capillary fabric can draw up the liquid solution from the container by capillary attraction to dispense a gaseous fluid into the atmosphere outside the container by means of evaporation of the liquid solution.

The invention relates to a dispensing system for dispensing a fluid into the atmosphere, wherein the system comprises a container for containing a liquid solution.

The invention also relates to a system comprising a rigid support and a capillary fabric.

The invention further relates to a method for using the dispensing system.

WO 2019/009702 discloses a dispensing system which uses a PVC bag and a capillary member to be connected to an outlet of the bag. The system provides excellent results at introducing a disinfecting gas into the atmosphere of for example an incubator (hatcher). However, installation of the bag in a room may be cumbersome and may in certain cases require additional means for installation or hanging the bag, such as a frame.

It is an object of the present invention to provide an improved dispensing system and/or a dispensing system having a relatively user-friendly and/or relatively fast installation.

At least one of these objects is achieved with a dispensing system as defined in claim 1.

The dispensing system for dispensing a fluid into the atmosphere comprises a container for containing a liquid solution, a rigid support and a capillary fabric, wherein a first section of the support is positioned inside the container through an opening of the container and a second section of the support is positioned outside the container, wherein the support is at least partially U-shaped or L-shaped by means of the second section of the support. The capillary fabric is attached around the first section and second section of the support, such that the capillary fabric can draw up the liquid solution from the container by capillary attraction to dispense a gaseous fluid into the atmosphere outside the container by means of evaporation of the liquid solution.

The support provides an improved dispensing system, because the first section of the support and the capillary fabric are positioned in the interior of the container such that there is a direct contact between the capillary fabric and the liquid solution inside the container. The capillary fabric may extend from the first section of the support in the direction of the bottom of the container. In this manner, a unsupported portion of the capillary fabric may be located close to or on the bottom of the container, or at least closer to the bottom than the first section of the support, such that as long as liquid solution is available, the system will work in a reliable manner. It is possible to provide an autonomous system by using capillary attraction, i.e. the system requires no pumps or the like or even electrical power to introduce a gaseous fluid into the atmosphere outside the container in a continuous manner. The system can be installed relatively fast and in a relatively user-friendly way, in that after the first section of the support with the capillary fabric is positioned inside the container through an opening of the container and the second section of the support with the capillary fabric is positioned outside the opening and not in the interior of the container, the system is operating or can be operated just by placing the container (filled with the liquid solution) at any desired position in a room to be treated with the gaseous fluid. The second section of the support provides or makes for an at least partially U-shaped or L-shaped support, which ensures that capillary fabric attached around the second section of the support is extending in a direction outside the container that is different from its direction within the container. The at least partially U-shaped or L-shaped support facilitates that the capillary fabric outside the container can draw up the liquid solution in an improved manner and/or through substantially the entire capillary fabric, such that an evaporation surface of the liquid solution outside the container can be increased in order to dispense the fluid into the atmosphere outside the container. The system with the at least partially U-shaped or L-shaped support has proven itself by excellent results of fluid dispensing into the atmosphere until the container is empty, in particular, when the liquid solution in the container is at a minimal level, and the path for the liquid solution through the capillary fabric to the opening is relatively long. The support may be J-shaped, or L-shaped or U-shaped, wherein a first leg of the support is substantially provided by the first section of the support. The J-shaped support may have an at least partially U-shaped second section of the support. In a L-shaped or a J-shaped support the second leg is shorter than the first leg, wherein the second leg is substantially provided by the second section of the support. The second section of the support is adapted to guide the capillary fabric in a radial direction with respect to the center line of the first section. The first section will in use extend substantially in a vertical direction. The second portion of the U-shaped support may comprise a bridging portion connecting the first leg with a second leg of the support. The bridging portion of the support may be a curved bridging portion or a straight bridging portion. An advantage of the U-shaped support is that by the capillary fabric attached around the second leg, i.e. the second leg is extending substantially parallel to the first leg, gravity may facilitate transport of the liquid solution towards the end of the capillary fabric located outside the container.

It is also an object of the present invention to provide an improved system (or assembly) to be used in the dispensing system as disclosed in this disclosure. This object is achieved with the system as claimed in claim 2. The system comprises a rigid support and a capillary fabric, wherein a first section of the support is configured to be positioned inside a container through an opening of the container and a second section of the support is configured to be positioned outside the container, wherein the support is at least partially U-shaped or L-shaped by means of the second section of the support, wherein the capillary fabric is attached around the first section and second section of the support, such that the capillary fabric can draw up the liquid solution from the container by capillary attraction to dispense a gaseous fluid into the atmosphere outside the container by means of evaporation of the liquid solution. The advantages of this system are already disclosed in this specification and are not repeated here for brevity reasons.

The container may be a portable container with a maximum volume of 75 dm³, preferably a maximum volume of 50 dm³ and/or a minimum volume of 0.5 dm³, preferably a minimum volume of 1 dm³. The container may be a plastic container, preferably a standard plastic container with a threaded cap for closing the opening, for example a 5, 10 or 20 dm³ standard plastic container, wherein the threaded cap of the container has been removed for insertion of the first section with the capillary fabric through the opening in the container. A container having at least one translucent or transparent indicator, has the advantage that an operator can easily determine the volume of liquid solution inside the container.

In one aspect, the opening of the container is positioned in a top wall of the container, wherein the first section of the support extends between the opening and a bottom wall of the container, wherein the length of the first section of the support is at least 70% of a height measured between the top wall and the bottom wall of the container. Such a configuration of the first section of the support ensures that the capillary fabric around the first section is guided by the rigid support towards the bottom of the container. In this manner, proper functioning of the system can be guaranteed independent of the slope of the surface on which the container bottom may be positioned.

In another aspect, the opening is defined by an inner edge, wherein the inner edge and/or the support and the capillary fabric are adapted to be in contact with each other to position and/or mount at least the first section of the support in a substantially vertical orientation. By providing contact between the edge of the opening and the support, the first section of the support can be mounted in an optimal position and/or vertical orientation for dispensing fluids by the system. In particular, when using standard containers, the dimensions of the opening are known, such that the edge surface defining the opening can provide the dimensions of a support for positioning and mounting the support in the container.

The system may be provided with a positioning tool in or over the opening of the container, wherein the positioning tool is adapted to receive and hold the support and the capillary fabric, to mount and/or position at least the first section of the support in a substantially vertical orientation. Such a positioning tool can be used in a user-friendly manner to mount and position at least the first section of the support in a substantially vertical orientation.

In a further aspect, the system further comprises a drip tray to be positioned below an end portion of the second section of the support, wherein the drip tray can be releasably connected to the container, preferably by clamping force.

Because of the at least partially U-shaped or L-shaped support, the end portion of the second section of the support, from a top view, is located outside the circumference of the container, wherein drips of liquid solution can be collected in a drip tray to avoid soiling of for example a floor of a room. By using the releasable connecting means (connector) of the drip tray, the drip tray can be automatically positioned in the correct position under the end portion of the second section of the support.

The drip tray may comprise a holder for receiving the end portion of the second section of the support. The holder of the drip tray can be used to hold the support, such that a stable support is obtained for guiding the capillary fabric attached around the support.

The invention will now be explained in more detail on the basis of an exemplary embodiment as shown in the appended drawings, in which:

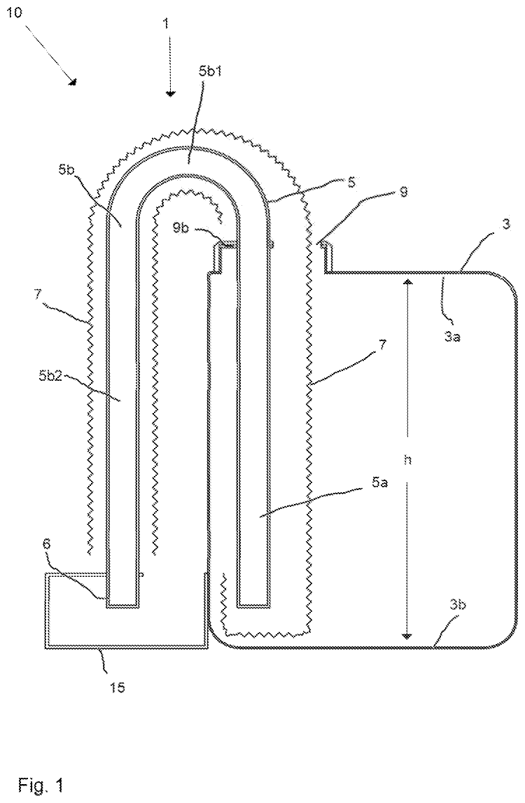

FIG. 1 shows a schematic cross-sectional view of a system for dispensing a fluid into the atmosphere;

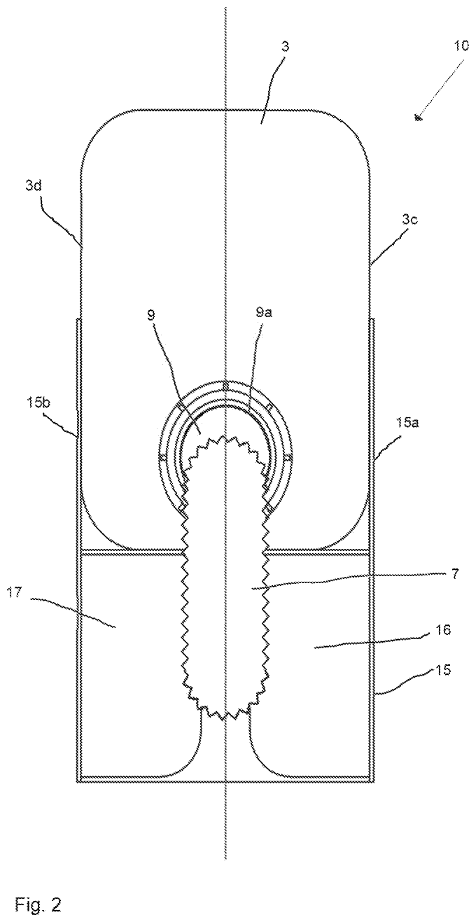

FIG. 2 shows a top view of the system shown in FIG. 1.

Like parts are indicated by the same numerals in the figures.

FIGS. 1 and 2 show a dispensing system 10 for dispensing a fluid into the atmosphere, wherein the system 10 comprises a container 3 for containing a liquid solution (not shown), a rigid support 5 and a capillary fabric 7. The rigid support 5 and the capillary fabric together form system 1 (FIG. 1).

The solution may be a disinfecting solution, for example as specified in WO 2019/009702.

A first section 5a of the support 5 is positioned inside the container 3 through an opening 9 of the container 3 and a second section 5b of the support 5 is positioned outside the container 3, wherein the support 5 is at least partially U-shaped by or thanks to the second section 5b of the support 5. The capillary fabric 7 is attached around the first section 5a and second section 5b of the support 5 such that the capillary fabric can draw up the liquid solution from the container 3 by capillary attraction to dispense a gaseous fluid into the atmosphere outside the container 3 by means of evaporation of the liquid solution.

The opening 9 of the container is positioned in a top wall 3a of the container 3, wherein the first section 5a of the support 5 has a length extending between the opening 9 and a bottom wall 3b of the container 3, wherein the length of the first section 5a of the support 5 is at least 70% of a height h measured between the top wall 3a and the bottom wall 3b of the container 3.

The capillary fabric 7 is made of a moisture-wicking material, such as a synthetic microfiber.

The container is a plastic container 3, such as a standard plastic container with a threaded cap (not shown) for closing the opening 9, for example during transport. The volume of the portable container is for example 5, 10 or 20 dm³. The container may be made of a translucent plastic material or have a translucent indicator section to indicate the volume of solution remaining in the container.

The opening is defined by an edge 9a, wherein the edge 9a and/or the support 5 and the capillary fabric 7 are adapted to be in contact with each other, to mount and position at least the first section of the support in a substantially vertical orientation.

The system 10, in particular the opening of the container may be provided with a positioning tool 9b to be mounted in or over the opening 9 of the container 3, wherein the positioning tool 9b is adapted to receive and hold the support 5 and the capillary fabric 7 to mount and position at least the first section 5a of the support in a substantially vertical orientation. It is also possible to use a cap which has been previously removed from the opening as a positioning tool (not shown). A cap sealing and closing the (non-standard or standard) container, for example during transport, may be used as positioning tool. After removal of the cap from the container, incisions can be made in the top of the cap to provide a cap with hingeable flanges through which the support and the capillary fabric may be passed by using manual force, such that at least the first section of the support can be mounted and positioned in a substantially vertical orientation inside the container.

In the embodiment shown in the figures a U-shaped support 5 has been shown, wherein the second portion 5b of the U-shaped support comprises a bridging portion 5b1 connecting the first leg 5a with a second leg 5b2 of the support, wherein the bridging portion of the support is curved. The first and second legs 5a, 5b2 have a corresponding length and extend parallel to each other.

As explained in this disclosure, the support may also be J-shaped or L-shaped (not shown).

In the U-shaped support 5 the first leg 5a of the support 5 is provided by the first section 5a of the support 5a. The second leg 5b2 may be shorter than the first leg 5a (not shown).

The system further comprises a drip tray 15 to be positioned below an end 6 portion of the second section 5b of the support 5, wherein the drip tray 15 is releasably connected to the container, preferably by clamping force, using two flanges 15a, 15b to be attached (e.g. clamped) to two opposing side walls 3c, 3d of the container 3.

The drip tray 15 comprises a holder for the end portion 6 of the second section of the support to provide a stable dispensing system 10. The holder may be a hole (not shown) provided by two plates 16, 17, in which the end portion 6 can be tight fitted. The dimensions of the dip tray may be different from the one shown, for example if a shorter leg 5b2 is provided than the one shown, the height of the dip tray may be higher in order to connect the holder to the end portion 6.

As can be seen in FIG. 1, the end portion 6 of the support 5 is not provided with capillary fabric, such that a relatively strong coupling can be made between the holder of the dip tray 15 and the end portion 6 of the support.

1-15. (canceled)

16. A dispensing system for dispensing a fluid into the atmosphere, comprising:
a container for containing a liquid solution;
a rigid support; and
a capillary fabric;
wherein:
a first section of the support is positioned inside the container through an opening of the container and a second section of the support is positioned outside the container;
the support is at least partially U-shaped or L-shaped by way of the second section of the support; and
the capillary fabric is attached around the first section and second section of the support, such that the capillary fabric is configured to draw up the liquid solution from the container by capillary attraction to dispense a gaseous fluid into the atmosphere outside the container by evaporation of the liquid solution.

17. The dispensing system according to claim 16, wherein:
the opening of the container is positioned in a top wall of the container;
the first section of the support has a length extending between the opening and a bottom wall of the container; and
the length of the first section of the support is at least 70% of a height measured between the top wall and the bottom wall of the container.

18. The dispensing system according to claim 16, wherein the capillary fabric is made of a moisture-wicking material.

19. The dispensing system according to claim 16, wherein the container is a portable container having a maximum volume of 75 dm³ and a minimum volume of 1 dm³.

20. The dispensing system according to claim 16, wherein:
the container is a plastic container including a threaded cap for closing the opening; and
the threaded cap is removable to insert the first section with the capillary fabric through the opening.

21. The dispensing system according to claim 16, wherein:
the opening is defined by an edge; and
the edge and/or the support and the capillary fabric are adapted to be in contact with each other to mount and/or position at least the first section of the support in a substantially vertical orientation.

22. The dispensing system according to claim 16, wherein:
the system includes a positioning tool in or over the opening of the container; and
the positioning tool is adapted to receive and hold the support and the capillary fabric to mount and/or position at least the first section of the support in a substantially vertical orientation.

23. The dispensing system according to claim 16, wherein:
the support is J-shaped, L-shaped, or U-shaped; and
a first leg of the support is provided by the first section of the support.

24. The dispensing system according to claim 23, wherein:
when the support is L-shaped or J-shaped, a second leg is shorter than the first leg; and
the second leg is provided by the second section of the support.

25. The dispensing system according to claim 23, wherein:
the second portion of the U-shaped support comprises a bridging portion connecting the first leg with a second leg of the support; and
the bridging portion of the support is a curved bridging portion or a straight bridging portion.

26. The dispensing system according to claim 16, further comprising a drip tray positionable below an end portion of the second section of the support, wherein the drip tray is releasably connectable to the container by a clamping force.

27. The dispensing system according to claim 26, wherein the drip tray comprises a holder configured to receive the end portion of the second section of the support.

28. The dispensing system according to claim 16, wherein the dispensing system is a power-free system configured to dispense a gaseous fluid into the atmosphere outside the container in a continuous manner.

29. The dispensing system according to claim 16, wherein the liquid solution is a disinfecting solution configured to disinfect at least one of a clean room, an incubator, and a cooler.

30. A system, comprising:
a rigid support; and
a capillary fabric;
wherein:
a first section of the rigid support is configured to be positioned inside a container through an opening of the container, and a second section of the support is configured to be positioned outside the container;
the rigid support is at least partially U-shaped or L-shaped by way of the second section of the rigid support; and
the capillary fabric is attached around the first section and the second section of the rigid support, such that the capillary fabric is configured to draw up liquid solution from the container by capillary attraction to dispense a gaseous fluid into the atmosphere outside of the container by evaporation of the liquid solution.

31. The system according to claim 30, wherein:
the opening of the container is positioned in a top wall of the container;
the first section of the support has a length extending between the opening and a bottom wall of the container; and
the length of the first section of the support is at least 70% of a height measured between the top wall and the bottom wall of the container.

32. The system according to claim 30, wherein the capillary fabric is made of a synthetic microfiber.
